(19) United States
(12) Patent Application Publication
(10) Pub. No.: US 2025/0107744 A1
(43) Pub. Date: Apr. 3, 2025

(54) EARLY DEMENTIA RISK ASSESSMENT DEVICE

(71) Applicant: HUNGKUANG UNIVERSITY, Taichung (TW)

(72) Inventors: YEN PO HUANG, Taichung (TW); ZONG MIN YANG, Taichung (TW); JUI CHEN HSU, Taichung (TW)

(21) Appl. No.: 18/476,811

(22) Filed: Sep. 28, 2023

(51) Int. Cl. A61B 5/00; A61B 5/22

(52) U.S. Cl. CPC A61B 5/4088; A61B 5/0002; A61B 5/225

(57) ABSTRACT

A device for assessing early dementia by testing gripping strength comprises a main member, a sensing member, and a control system. The main member has a display portion and a gripping portion. The sensing member is connected to the gripping portion to be pressed by a subject to generate a sensing signal. The control system is provided in the main member and having a control unit, a timer, a grip sensing module, and a determining module. The timer records a pressing time of the subject. The grip sensing module receives the sensing signal from the sensing members and generates a gripping strength signal according to the sensing signals. The determining module receives the pressing time and the gripping strength signal, and generates an assessing result by comparing the gripping strength signal with a gripping strength threshold and comparing the pressing time with a time threshold.

### BACKGROUND OF THE INVENTION

### 1. Technical Field

The present invention relates to an assessment device, and more particularly to a device, which can assess a risk of early dementia through grip strength.

### 2. Description of Related Art

The world has entered an aging society. Some elder people are suffering from dementia due to reduced activity and exercise. There are studies showing that people with sarcopenia are particularly prone to dementia. In addition to the muscle-building effect, it also helps to prevent dementia.

A prior art taught a gripping device for training cognition, which includes a carrier member, a resistance source, a press sensing module, a hint module, and a data processing module. A subject holds the carrier member, the data processing module controls the hint module to light up, which is a sign to inform the subject to do the grip training and a cognition training. The press sensing module senses the pressure of each finger, but the conventional device didn't do any examining of the pressure and the cognition.

Another prior art taught a robot with a sensing unit, which has a plurality of grip sensors mounted on a hand of the robot. The grip sensors sense pressures while a subject holds the hand of the robot, and transmits the pressure data to a grip data base. An examining unit is provided to examine the pressure data to determine the subject's brain function. However, the sensing unit is provided on the hand of the robot, so the sensors are very unstable in pressure detection.

Another prior art taught a method and a system for assessing intra-individual reaction variability, in which a screen shows a horizontal band shifting in a vertical direction, and a subject holds a grip dynamometer and exerts it control a vertical band on the screen according to the band. The data of the positions of the vertical band and the horizontal band and the pressure data of the grip dynamometer are collected and examined to assess the subject for mild traumatic brain injury (MTBI) or other cognitive impairment (eg, brain injury, concussion, dementia). However, this convention test is done only when the grip dynamometer accompanies the visual movement.

### BRIEF SUMMARY OF THE INVENTION

In view of the above, the primary objective of the present invention is to provide a device for assessing early dementia through a grip strength test.

In order to achieve the objective of the present invention, A device for assessing early dementia by testing gripping strength comprises a main member, a sensing member, and a control system. The main member has a display portion and a gripping portion, wherein the display portion has a screen to show information, and the gripping portion is connected to the display portion. The sensing member is connected to the gripping portion to be pressed by a subject to generate a sensing signal. The control system is provided in the main member and having a control unit, a timer, a grip sensing module, and a determining module. Wherein, the timer is electrically connected to the control unit to record a pressing time of the subject pressing the sensing member. Wherein, the grip sensing module is electrically connected to the control unit and the sensing member; the grip sensing module receives the sensing signal from the sensing members and generates a gripping strength signal according to the sensing signals. Wherein, the determining module is electrically connected to the control unit to receive the pressing time and the gripping strength signal. The determining module then generates an assessing result by comparing the gripping strength signal with a gripping strength threshold and comparing the pressing time with a time threshold, and the assessing result is shown on the screen.

In an embodiment, the gripping portion is provided with a plurality of grooves, and the sensing member has a plurality of buttons mounted on sidewalls of the grooves respectively to be pressed by fingers of the subject.

In an embodiment, the gripping portion has an opening, in which a shifting member and a spring are received; the spring has opposite end connected to the shifting member and the sensing member whereby the shifting member is moved by the subject to compress the spring and make the sensing member generate the sensing signal.

In an embodiment, the gripping portion has a shifting member kept a predetermined distance from the gripping portion and a spring having opposite ends connected to the sensing member and the shifting member whereby the shifting member is moved by the subject to compress the spring and make the sensing member generate the sensing signal.

In an embodiment, the gripping portion has two connecting members and, a middle member, and two handles; the connecting members each has a first end connected to the display portion and a second end opposite to the first end; the sensing member has two sensors connected to the second ends of the connecting members respectively; the middle member has two poles connected to the handles and resting against the sensors respectively whereby the handles is held by the subject and pressed to have the sensors being pressed by the poles and generating the sensing signal accordingly.

In an embodiment, the middle member has loops, and the poles are connected to the loops.

In an embodiment, the control system further includes a setting module mounted on the display portion; the setting module has a power button and two setting buttons, which are electrically connected to the control unit.

In an embodiment, the control system further includes a storage unit, in which personal information, the time threshold, the gripping strength threshold, the pressing time and the gripping strength signal are stored; the storage unit is electrically connected to the control unit.

In an embodiment, the control system further includes a communication module to transmit the assessing result out.

In an embodiment, wherein the communication module transmits the assessing result in a wireless way.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a perspective view of a first preferred embodiment of the present invention;

FIG. 2 is another perspective view of the first preferred embodiment of the present invention;

FIG. 3 is a sectional view of the first preferred embodiment of the present invention;

FIG. 4 is a block diagram of the first preferred embodiment of the present invention;

FIG. 5 is a flow chart of the first preferred embodiment of the present invention;

FIG. 6 is a perspective view of a second preferred embodiment of the present invention;

FIG. 7 is a top view of the second preferred embodiment of the present invention, showing the spring and the sensor inside;

FIG. 8 is a perspective view of a third preferred embodiment of the present invention;

FIG. 9 is a front view of the third preferred embodiment of the present invention; and

FIG. 10 is a perspective view of a fourth preferred embodiment of the present invention.

### DETAILED DESCRIPTION OF THE INVENTION

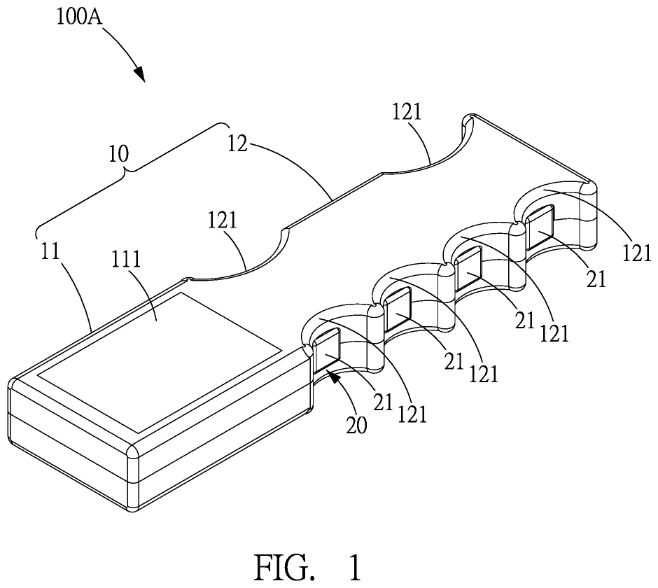

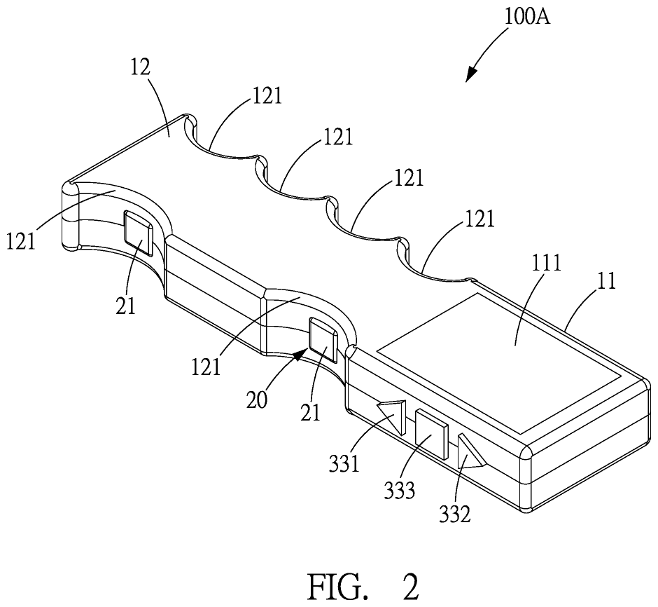

As shown in FIGS. 1-5, a device 100A for assessing early dementia of the first preferred embodiment of the present invention includes a main member 10, a sensing member 20, and a control system 30.

The main member 10 has a display portion 11 and a gripping portion 12. The display portion 11 has a screen 111 at a front side thereof to show information. The gripping portion 12 is connected to the display portion 11 and has a plurality of grooves 121 on lateral sides thereof. In the present embodiment, the gripping portion 12 has four of the grooves 121 on a lateral side and two of the grooves 121 on an opposite lateral side. A subject may put his/her finger in the grooves 121 to hold the gripping portion 12 in opposite directions.

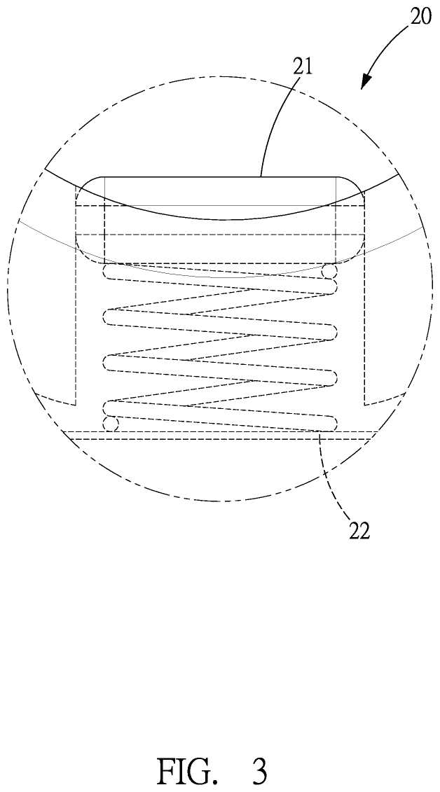

As shown in FIG. 3, the sensing member 20 has a plurality of buttons 21 and sensors 22. The buttons 21 are mounted on sidewalls of the grooves 121, and the sensors 22 are connected to the buttons 21 respectively to generate sensing signals N when the subject holds the gripping portion 12 and exert the buttons 21 by the fingers. In the present invention, there are six buttons 21 in the six grooves 121 respectively. Therefore, there are six sensing signals N being generated by the sensors 22 when the subject holds the gripping portion 12 and exerts.

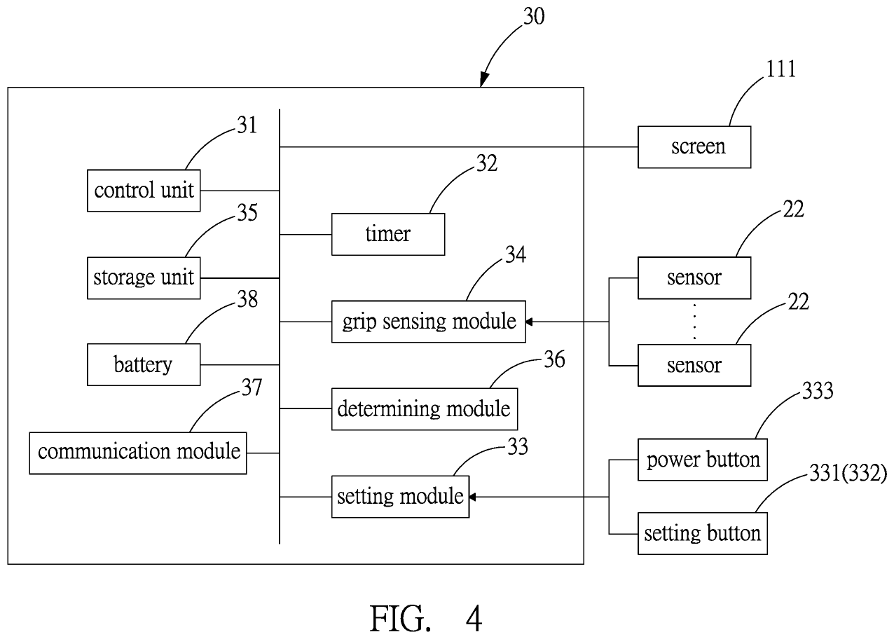

The control system 30 is received in the display portion 11 and electrically connected to the screen 111. As shown in FIG. 4, the control system 30 includes a control unit 31, a timer 32, a setting module 33, a grip sensing module 34, a storage unit 35, a determining module 36 and a communication module 37.

The control unit 31, such as MCU, RAM, or ROM, is provided to execute all the processes and operations.

The timer 32 is electrically connected to the control unit 31 to record a pressing time t of the subject pressing the sensing member 20. The timer 32 starts as the fingers touching the sensing member 20, and stops when the fingers leave the sensing member 20.

The setting module 33 is provided on the display portion 11 and electrically connected to the control unit 31. In the present embodiment, the setting module 33 includes two triangular setting buttons 331 and 332 to be manipulated to set or change a gripping strength threshold P and a time threshold T, and the current gripping strength threshold P and the time threshold T are shown on the screen 111. The setting module 33 further includes a power button 333 between the setting buttons 331 and 332 to turn on/off a battery 38 which provides the device 100A power.

The grip sensing module 34 is electrically connected to the control unit 31 and the sensing member 20 to receive the sensing signals N from the sensing member 20 and convert them into a gripping strength signal p, and then the gripping strength signal p is transmitted to the control unit 31. It is noted that the gripping strength signal p is a sum of the sensing signals N of the sensing members 20. In the present embodiment, the gripping strength signal p and the gripping strength threshold P are in the form of voltage.

The storage unit 35 is electrically connected to the control unit 31 to store personal information (such as age and sex), the time threshold T, the gripping strength threshold P, and the gripping strength signal p and the pressing time t of each test.

The determining module 36 is electrically connected to the control unit 31 to generate an assessing result according to the time threshold T, the gripping strength threshold P, the gripping strength signal p and the pressing time t. In the present embodiment, a predetermined assessment algorithm is used to calculate all the parameters as described above. The assessing result is shown on the screen 111 to give the subject a risk of getting early dementia.

A communication module 37 is a wireless communication module in the present embodiment, which is electrically connected to the control unit 31 to transmit the assessing result to a smartphone (not shown) in a wireless way.

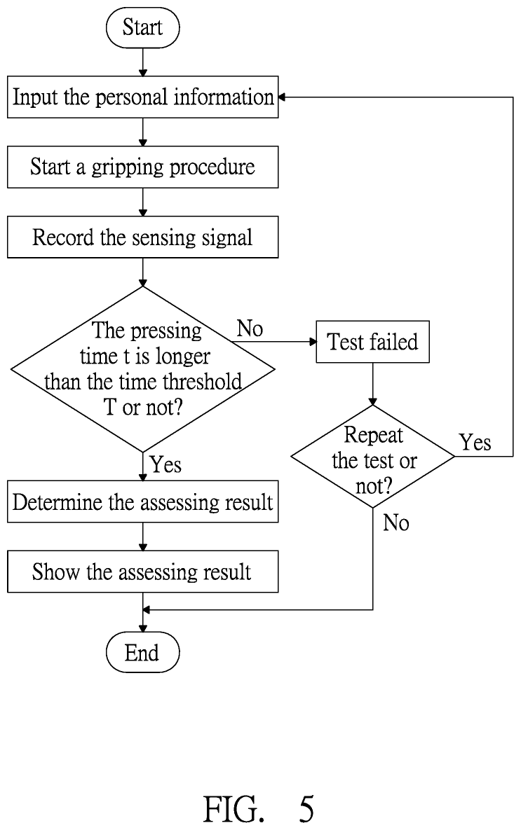

As shown in FIG. 5, to proceed an assessment test by the device 100A of the first preferred embodiment of the present invention, the power button 333 is turned on, and then proceed a setting procedure, including inputting the personal information (sex, age), the gripping strength threshold P and the time threshold T by manipulating the setting buttons 331 and 332. The gripping strength threshold P and the time threshold T are obtained from numerous medical studies, and they are related to sex and age. After all the data are set, it may start a gripping procedure. In the gripping procedure, the subject holds the gripping portion 12 and rests his/her fingers in the corresponding grooves 121, and then the subject is asked to press the buttons 21 of the sensing member 20 according to the commands on the screen 111. The sensing signals N are recorded and compared with the gripping strength threshold P when the pressing times t is longer than the time threshold T. The assessment test is successful if the sensing signals N are greater than the gripping strength threshold P, otherwise the test is failed. If a test is determined failed and the subject wants to repeat the test, he/she has to repeat the setting procedure and the gripping procedure again. After the assessment test is completed, no matter it is successful or failed, an assessing result is shown on the screen 111. The assessing result shows “High Risk of Dementia”, “Fail”, or “Bad” on the screen 111 when the assessment test is failed. On the contrary, the assessing result shows “Low Risk of Dementia”, “Good”, or “Not Bad” on the screen 111 when the assessment test is successful.

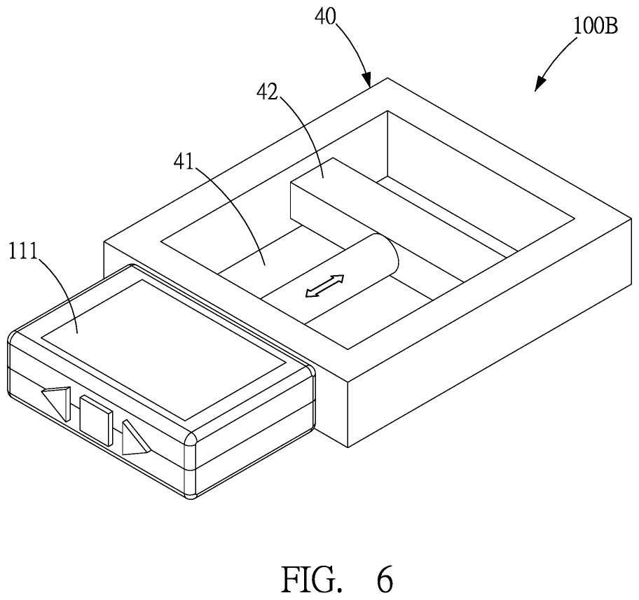

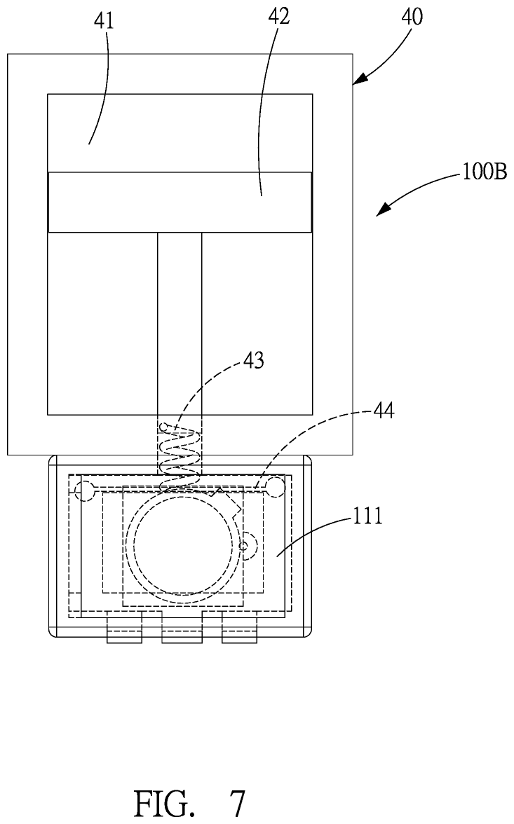

FIG. 6 and FIG. 7 show a device 100B for assessing early dementia of the second preferred embodiment of the present invention, which is substantially the same as the device 100A of the first preferred embodiment, except that

A gripping portion 40 is a rectangular frame in the present embodiment, which has an opening 41 at a center thereof, a shifting member 42 and a spring 43. The shifting member 42 is a T-shaped member received in the opening 41 for movement. The spring 43 has opposite ends connected to the shifting member 42 and a sensor 44. A subject may hold the gripping portion 40 and grips the shifting member 42 by fingers. The sensor 44 may generate the sensing signal N when the subject exerts the shifting member 42. The testing procedures are the same as the first preferred embodiment, so I do not describe again.

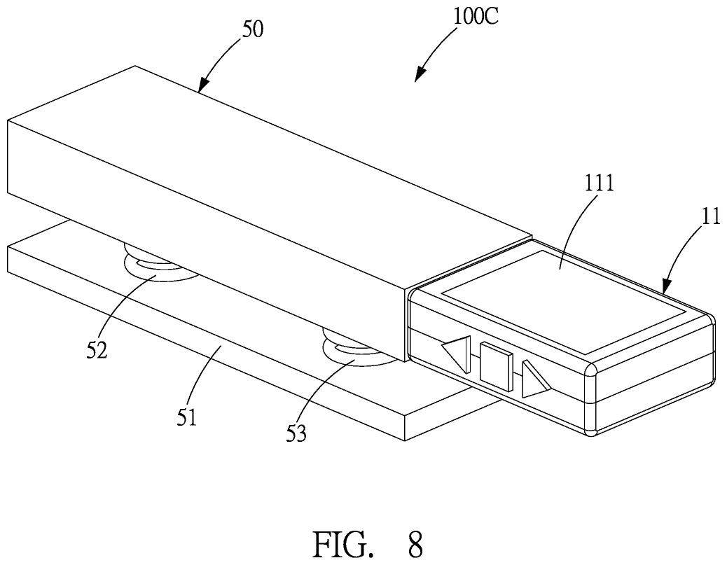

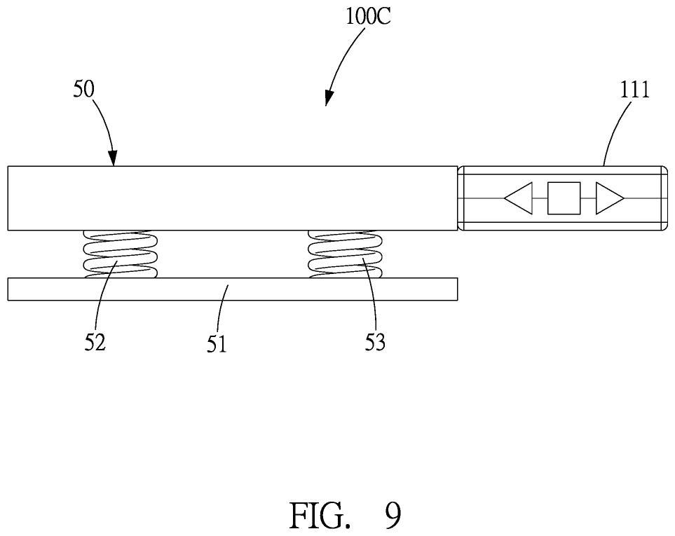

FIG. 8 and FIG. 9 show a device 100C for assessing early dementia of the third preferred embodiment of the present invention, which is substantially the same as the device 100B of the second preferred embodiment, except that

A gripping portion 50 is a cuboid, and a shifting member 51 is a plate. The shifting member 51 is movably connected to the gripping portion 50 via two springs 52 and 53. The shifting member 51 is kept a predetermined distance from the gripping portion 50 in an initial position, and is moved toward to the gripping portion 50 to compress the springs 52 and 53 when a subject holds the gripping portion 50 and move the shifting member 51 by fingers. The testing procedures are the same as the first preferred embodiment, so I do not describe again.

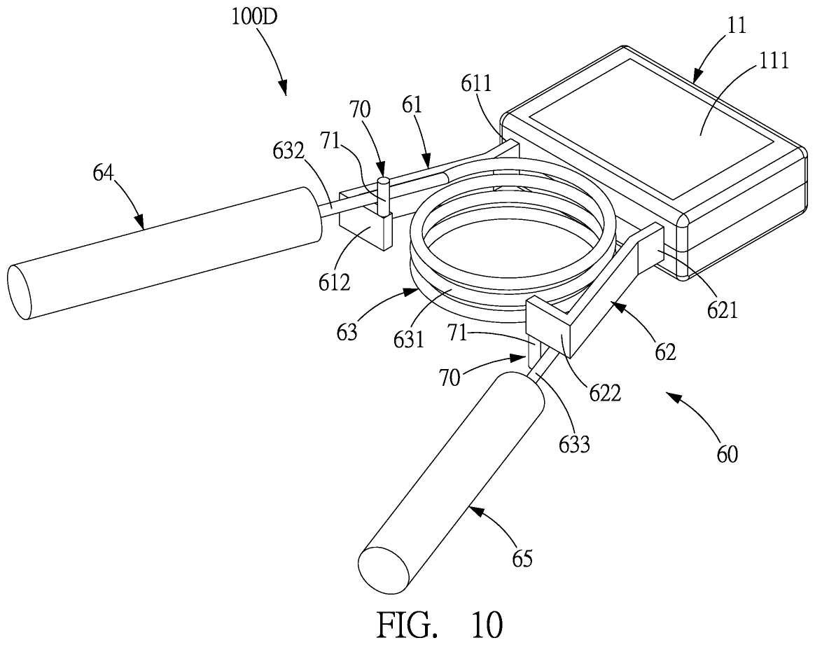

FIG. 10 shows a device 100D for assessing early dementia of the fourth preferred embodiment of the present invention, which is substantially the same as the device 100A of the first preferred embodiment, except that

A gripping portion 60 has two connecting members 61 and 62, a middle member 63, and two handles 64 and 65. The connecting members 61 and 62 each has a first end 611 and 621 connected to the displace portion 11 and a second end 612 and 622 as a free end to be moved toward to each other. the middle member 63 has plural loops 631 and two poles 632 and 633 connected to handles 64 and 65 respectively. Two sensing members 70 each has a sensor 71. The sensors 71 are connected to the second ends 612 and 622 of the connecting members 61 and 62, and rest against the connecting members 61 and 62 respectively. A subject may hold the handles 64 and 65 by his/her palm and exerts to move the handles 64 and 65 inwardly to make the sensors 71 to be pressed by the connecting members 61 and 62 and get the sensing signals N. The testing procedures are the same as the first preferred embodiment, so I do not describe again.

In conclusion, the present invention provides several gripping portions 12, 40, 50, and 60 to be manipulated by subjects for detecting the gripping strength signals p so as to assess a risk of early dementia by comparing the gripping strength signals p with the gripping strength threshold P and comparing the pressing times t with the time threshold T. The assessing result is shown on the screen 111 to inform the subject.

It must be pointed out that the embodiments described above are only some preferred embodiments of the present invention. All equivalent structures which employ the concepts disclosed in this specification and the appended claims should fall within the scope of the present invention.

What is claimed is:

1. A device for assessing early dementia by testing gripping strength, comprising:
a main member having a display portion and a gripping portion, wherein the display portion has a screen to show information, and the gripping portion is connected to the display portion;
a sensing member connected to the gripping portion to be pressed by a subject to generate a sensing signal;
a control system provided in the main member and having a control unit, a timer, a grip sensing module, and a determining module;
wherein the timer is electrically connected to the control unit to record a pressing time of the subject pressing the sensing member;
wherein the grip sensing module is electrically connected to the control unit and the sensing member; the grip sensing module receives the sensing signal from the sensing members and generates a gripping strength signal according to the sensing signals;
wherein the determining module is electrically connected to the control unit to receive the pressing time and the gripping strength signal; the determining module generates an assessing result by comparing the gripping strength signal with a gripping strength threshold and comparing the pressing time with a time threshold, and the assessing result is shown on the screen.

2. The device of claim 1, wherein the gripping portion is provided with a plurality of grooves, and the sensing member has a plurality of buttons mounted on sidewalls of the grooves respectively to be pressed by fingers of the subject.

3. The device of claim 1, wherein the gripping portion has an opening, in which a shifting member and a spring are received; the spring has opposite end connected to the shifting member and the sensing member whereby the shifting member is moved by the subject to compress the spring and make the sensing member generate the sensing signal.

4. The device of claim 1, wherein the gripping portion has a shifting member kept a predetermined distance from the gripping portion and a spring having opposite ends connected to the sensing member and the shifting member whereby the shifting member is moved by the subject to compress the spring and make the sensing member generate the sensing signal.

5. The device of claim 1, wherein the gripping portion has two connecting members and, a middle member, and two handles; the connecting members each has a first end connected to the display portion and a second end opposite to the first end; the sensing member has two sensors connected to the second ends of the connecting members respectively; the middle member has two poles connected to the handles and resting against the sensors respectively whereby the handles is held by the subject and pressed to have the sensors being pressed by the poles and generating the sensing signal accordingly.

6. The device of claim 5, wherein the middle member has loops, and the poles are connected to the loops.

7. The device of claim 1, wherein the control system further includes a setting module mounted on the display portion; the setting module has a power button and two setting buttons, which are electrically connected to the control unit.

8. The device of claim 1, wherein the control system further includes a storage unit, in which personal information, the time threshold, the gripping strength threshold, the pressing time and the gripping strength signal are stored; the storage unit is electrically connected to the control unit.

9. The device of claim 1, wherein the control system further includes a communication module to transmit the assessing result out.

10. The device of claim 9, wherein the communication module transmits the assessing result in a wireless way.
